(19) United States
(12) Patent Application Publication
(10) Pub. No.: US 2010/0273253 A1
(43) Pub. Date: Oct. 28, 2010

(54) DYNAMIC SYSTEMS FOR CULTURING CELLS IN 3D SUPPORTS

(76) Inventors: Joao Manuel Teixeira de oliveira, Malosinhos (PT); Adriano José De Abreu Pedro, Braga (PT); Roi Luis Goncalves Dos Reis, Porto (PT)

Correspondence Address: PATENT DOCKET CLERK;COWAN, LIEBOWITZ & LATMAN, P.C., 1133 AVENUE OF THE AMERICAS, NEW YORK, NY, 10036, US

(21) Appl. No.: 12/808,291

(22) PCT Filed: Dec. 18, 2008
(86) PCT No.: PCT/IB2008/003572

§ 371 Date: Jun. 15, 2010

(30) Foreign Application Priority Data
Dec. 20, 2007 (PT) 103906

(51) Int. Cl. C12M 1/12

(52) U.S. Cl. 435/297.1

(57) ABSTRACT

The present invention refers to the conception process of new dynamic systems for cell culture in 3D supports appropriate to its growing. These are constituted by:—cylindrical container (1) exhibiting a terminal area with a truncated inverted cone shape (2) in the lower internal zone;—cylindrical screw lid (4) the upper external zone of which has a cylindrical tube (7) that penetrates the whole extension of the lid, also containing a coupling central hole (6);—screw cap (10) containing a filter (11) that fits in the cylindrical tube (7) of the lid (4); hexagonal shaft (12) with an insertion for the derivations in the lower terminal part (part ‘E’);—Derivation in the form of tweezers forming a gripping tool that sustains the 3D supports. These new systems allow a yield increase in biological material costs, laboratory consumable and time spent by technical specialized staff.

### OBJECT OF INVENTION

The present invention refers to a new dynamic system of culture of different cell types in three-dimensional supports appropriate to its cultivation.

### STATE OF THE ART

Several studies demonstrated that cellular proliferation increases in response to mechanical forces applied through the movement of fluids, when compared to static conditions of culture.

In this way, dynamic systems are frequently used in cell culture, being commonly designated as bioreactors (JP62171680 and JP62000274).

They are used in the cultivation of different cell types including vegetable cells, and animal cells such as hematopoietic stem cells (Kwon, J., Kim, B.-S., Kim, M.-J., and Park, H.-W., Suspension culture of hematopoietic stem cells in stirred bioreactors, Biotechnology Letters 25 (2), 179-182, 2003 and Nielsen, L. K., Bioreactors for Hematopoietic Cell Culture, 1999, pp. 129-152) and neuronal stem cells (Michael S. Kallos, L. A. B., Inoculation and growth conditions will be cell high-cell-density expansion of mammalian neural stem cells in suspension bioreactors, 1999, pp. 473-483).

Dynamic culturing systems are also used for the cultivation of cells in three-dimensional supports having in mind the regeneration of several types of tissues (e.g. bone, cartilage, skin). Different dynamic scenarios may include perfusion environments, simulated microgravity, or intermittent compression (Martin, I., Wendt, D., and Heberer, M., The rolls of bioreactors in tissue engineering, Trends in Biotechnology 22 (2), 80-86, 2004).

One of these systems consists of a container with a point of vortex that is responsible for the constant recirculation of culture medium containing a suspension of cells. Three-dimensional supports appropriate for the cultivation of these cells are immersed in the medium and are expected to function as a substrate in which cells can grow. (Todd M. Upton, J. T. F., Sep. 22, 2000, Cell culture spinner flasks). The constant functioning of this system shall progressively lead to the colonization of these supports by the cells being the cell-material hybrid structure intended to be used in further stages.

A major drawback of these traditional systems is the use of a significant volume of culture medium, which proportionally correlates with the number of cells that will have to be obtained to achieve a constant cellular concentration. Although greatly depending on the cell type, reaching an extraordinarily high number of cells often imposes additional efforts in terms of costs and human resources. It should be mentioned that the cells with higher impact and relevance in the tissue engineering field are obtained from primary cultures, which frequently demand specific conditions and parameters of culture. This feature, in association with the often reduced number of cells obtained after isolation may restrict the experimental design and condition the scientific analysis. Even if this hurdle is surpassed, the time of proliferation in two-dimensional culture (2D) necessary to reach the desired number of cells persists on being a delaying factor. Another disadvantage of the traditional system relates to the perforation that has to be conducted on the samples, in order to assure their sustenance. This occurrence greatly limits the type of samples that can be used and in addition alters their 3D structure by creating a drill.

### BRIEF DESCRIPTION OF THE FIGURES

FIG. 1 represents the cylindrical container that will delimit the physical space where the culture systems will be included;

FIG. 2 represents the screw lid of the container represented in FIG. 1;

FIG. 3 represents the screw capsule containing a filter responsible for controlling the entry and exit of particles in the system;

FIG. 4 represents the shaft responsible for supporting the derivations;

FIG. 5 represents a derivation for sustaining the supports;

FIG. 6 represents an internal view of the assembly of the system;

FIG. 7 represents an external view of the assembly of the system

### DESCRIPTION OF THE INVENTION

The new dynamic system for culturing cells in three-dimensional supports described in the present invention is constituted by 5 parts, namely:

### Part A—Cylindrical Container

### Part B—Lid

### Part C—Capsule

### Part D—Shaft

### Part E—Derivation

In this new system the previously reported situations (see State of the art page 1) are avoided due to 2 factors: 1) reduction of the volume of cell suspension used; and 2) the existence of a gripping mechanism able to support all kinds of structures. The first, results from the design and manufacture of part “A”, which exhibits a terminal area with a truncated inverted cone shape in the lower internal zone that reduces the volume of culture medium and number of cells needed when compared to the traditional systems. Therefore, the time and resources applied will be decrease significantly making this system a more viable and efficient option.

The second factor clearly distinguishes this new system from the traditional ones due to the adaptability to different types of three-dimensional (3D) supports that it provides. In the traditional systems, the cell supports used must resist perforation, since their sustenance is assured by a fixed steel wire that perforates the 3D structure completely. In the new system described, a plastic fixed vein constituted by several derivations in its lower part is responsible for the sustenance of the cell supports avoiding perforation.

Each derivation has two other derivations in its lower end that together form a gripping tool responsible for supporting the 3D structure. This gripping tool can easily adapt to the necessary compressive effort that is able to guarantee the proper sustaining of the supports. In this way, the perforation of samples that alters their initial morphology is avoided. Additionally, the use of 3D supports of various shapes and sizes is enabled, increasing the types of samples that can be placed in these dynamic systems.

This invention also simplifies the handling and insertion of the cell supports in the systems. The ease of gripping and assembly conferred by the derivations avoids the skilled handling that has to be performed in the traditional systems.

In conclusion, these new dynamic systems for culturing cells in 3D supports allow a reduction in the number of cells needed, permit the use of a wide variety of 3D supports and greatly facilitate handling and sample manipulation.

The next table summarizes the main advantages of the new system when compared to the traditional ones.

| TRADITIONAL SYSTEMS | NEW SYSTEM |
|---|---|
| Uses a higher volume of | Uses less volume of culture |
| culture medium | medium |
| High number of cells is | Less number of cells is |
| needed | needed |
| More time spent and more | Less time spent and less |
| resources | resources |
| The 3D supports must resist | Applicable to different |
| mechanically to perforation | types of 3D supports |
| Difficult handling for inserting | Easy handling for inserting |
| samples into the system | samples into the system |

For a clearer understanding of the invention, a detailed explanation of the developed parts is presented below in a non-limitative fashion with associated illustrative figures.

### DETAILED DESCRIPTION OF THE DRAWINGS

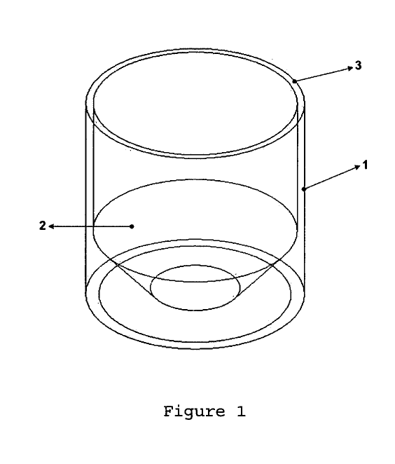

FIG. 1 represents the part “A” comprising a cylindrical container (1) processed by injection molding in polycarbonate or polypropylene, although this can be injected in another type of thermoplastic material. The lower external zone is flat and parallel to the plan of the ground. The lower internal zone contains a terminal area with a truncated inverted cone shape (2) that does not penetrate the base of the tube, being this ending executed in a parallel plan to the lower external surface. The upper external zone is flat and parallel to the plan of the ground. The upper internal zone contains a screw (3) for assembling with part B.

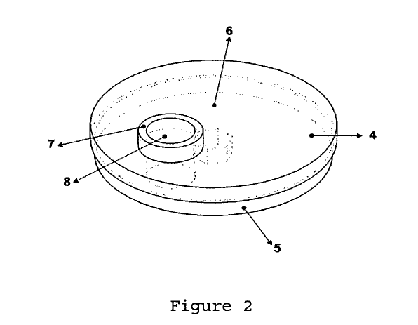

FIG. 2 represents the part “B” that consists of a cylindrical screw cap (4) processed by injection molding in polycarbonate or polypropylene, although this can be injected in another type of thermoplastic material. The lower zone contains a screw for assembling with the upper internal zone (5) of the part “A”. The circumferential lower plan, flat and parallel to the plan of the ground, which belongs to the lower zone of the part “B”, contains a central orifice responsible (6) for assembling with the upper zone of the part “D”. The upper external zone of part “B” contains a region for assembling the capsule (part “C”) (7) that perforates the whole part, enabling gas exchanges through the part “B”, after the complete assembly of the system. This region presents a screw in the upper external zone (8), responsible for assembling the part “C”.

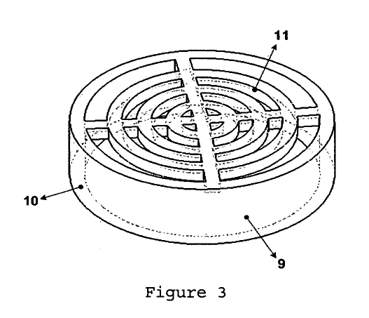

FIG. 3 is a representation of the part “C” that consists of a screw cap (9) injected in polypropylene. The lower internal zone of the part “C” contains an end screw (10) responsible for the assembly in the part “B”. The upper central zone has a circumferential filter (11) of hydrophobic cellulose that substitutes the polypropylene. This filter is responsible for controlling the entry and exit of particles based on their size between the internal part of the part “A” and the external environment, thereby reducing the risks of contamination.

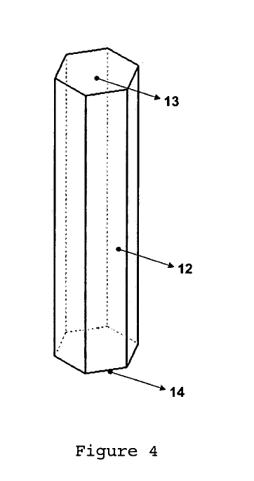

FIG. 4 is a representation of part “D” that consists of a plastic shaft (12) with six derivations in the terminal lower part. The upper zone of the main shaft (13) fits with the central orifice of the circumferential lower plan of part “B”. The lower region of the main shaft is hexagonal, having in each face an insertion (14) for each one of the derivations (part “E”).

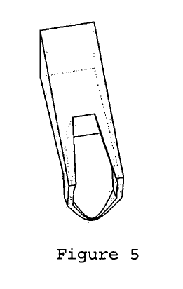

FIG. 5 represents part “E” that is molded by injection in the form of tweezers that fit in each one of the faces of the hexagonal shaft (part “D”). This part will sustain the supports for tissue engineering used for each application.

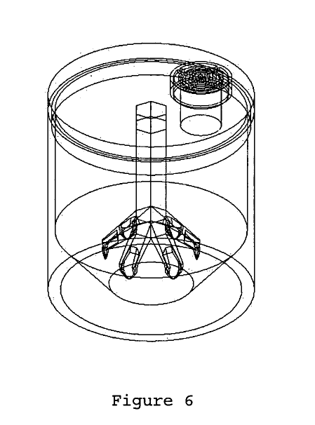

FIG. 6 represents an internal view of the assembly of the system.

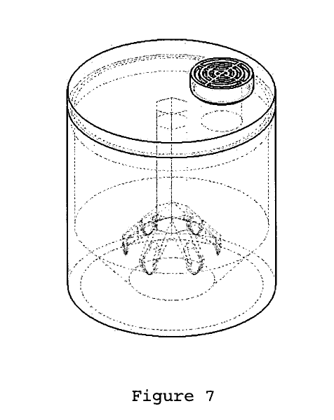

FIG. 7 represents an external view of the assembly of the system.

1. Dynamic systems of cell culture in three-dimensional supports, comprising:
a cylindrical container with a terminal area possessing a truncated inverted cone shape in the lower internal zone and the upper zone for screwing the lid that will delimit the physical space where the culture systems will be included, together with the lid;
a cylindrical lid with a screw for part “A”, the upper external zone of which contains a cylindrical tube that perforates the whole extension of the lid, enabling the existence of gas exchanges after the complete assembly of the system delimiting the physical space where the culture systems will be included at the level of the top of part “A”; this region has a central orifice of assembly for part “D”;
a screw cap containing a filter responsible for controlling the entry and exit of particles in the system based on their size and that screws in the cylindrical tube of part “B”;
hexagonal shaft with an insertion in each surface in the terminal lower part for the derivations (part “E”), which shaft contains a connection zone in its top, which will form the connection with part “B”, being supported in this way.
derivations in the form of tweezers, which fit in each surface of the hexagonal shaft to assure the appropriate sustaining of the several 3D supports.

2. Dynamic cell culture systems of three-dimensional supports, according to claim 1 wherein they exhibit a terminal area with a truncated inverted cone shaped in the lower internal zone of the container (1), reducing the volume of culture medium and number of cells used, when compared to the traditional systems.

3. Dynamic cell culture systems of three-dimensional supports, according to claim 1 wherein they are adaptable to different types of three-dimensional supports, being the fixed plastic shaft constituted by several derivations in its lower part; these derivations are responsible for sustaining the supports for cell growth and development and for avoiding the perforation of these same supports, enabling thereby that different types of these can be used.

4. Dynamic cell culture systems of three-dimensional supports, according to claim 1 wherein each derivation has two derivations in its end forming a gripping tool, which is responsible for sustaining the 3D biological supports; this gripping tool can easily adjust to the compression effort necessary to guarantee the efficient sustaining of the 3D biological supports.

5. Dynamic cell culture systems of three-dimensional supports, according to claim 3, wherein each derivation has two derivations in its end forming a gripping tool, which is responsible for sustaining the 3D biological supports; this gripping tool can easily adjust to the compression effort necessary to guarantee the efficient sustaining of the 3D biological supports.
